(19) United States
(12) Patent Application Publication
(10) Pub. No.: US 2006/0121049 A1
(43) Pub. Date: Jun. 8, 2006

(54) Chimeric recombinant protein and in vitro diagnosis

(76) Inventor: Odile Letourneur, SAINT FOY LES LYON (FR)

Correspondence Address: OLIFF & BERRIDGE, PLC, P.O. BOX 19928, ALEXANDRIA, VA, 22320, US

(21) Appl. No.: 10/526,765

(22) PCT Filed: Sep. 15, 2003
(86) PCT No.: PCT/FR03/02712

§ 371 Date: Mar. 7, 2005

(30) Foreign Application Priority Data
Sep. 17, 2002 (FR) 02/11485

(51) Int. Cl. A61K 39/21; C07H 21/04; C07K 14/16; C07H 21/02; C12N 15/867

(52) U.S. Cl. 424/188.1; 435/69.3; 435/456; 435/325; 530/350; 536/23.72

(57) ABSTRACT

The invention relates to a recombinant DNA encoding a chimeric recombinant protein, comprising at least two first nucleotide fragments each encoding an epitope region of the HIV-1 virus group M or group O or of the HIV-2 virus, at least a second nucleotide fragment encoding a linking region, at least a third nucleotide fragment encoding an attaching region, characterized in that each first nucleotide fragment encodes at least one immunodominant region of the gp120 glycoprotein of HIV-1, of the gp41 glycoprotein of HIV-1 group M, of the gp41 glycoprotein of HIV-1 group O or of the gp36 glycoprotein of HIV-2. The invention also relates to a recombinant chimeric protein encoded by the DNA defined above, and also to the use of said DNA and/or of said recombinant protein for in vitro diagnosis.

The present invention relates to a chimeric recombinant protein, to a DNA encoding said chimeric recombinant protein, and also to the use of this chimeric recombinant protein for the in vitro diagnosis of diseases related to a virus, more particularly the HIV-1 and/or HIV-2 virus.

Early diagnosis of the presence of a virus in the organism is essential in order, firstly, to prevent propagation of the virus by patients who do not yet know that they are seropositive and, secondly, to provide these patients with an appropriate treatment in order to push back the time at which symptoms appear.

In the case of AIDS (acquired immunodeficiency syndrome), which is the result of infection with the HIV-1 (human immunodeficiency virus-1) or HIV-2 (human immunodeficiency virus-2), retroviruses, the primary infection is followed by an asymptomatic period, of variable duration, before the disease evolves, in most patients, into AIDS, characterized by the appearance of infections with opportunistic microorganisms, of tumors and of neurological manifestations, and there must be early diagnosis of the presence of the HIV virus in the organism whether the patient was initially infected with HIV-1 or with HIV-2.

Most of the diagnostic tests marketed are based on an antigen-antibody reaction directed against certain viral proteins, such as the transmembrane protein of the viral envelope. In the case of HIV-1, the envelope proteins are derived from the env gene, which encodes a precursor glycoprotein having a molecular weight of 160 000 daltons, called gp160. gp160 is then cleaved into two viral proteins of the envelope, gp120 and gp41. In the case of HIV-2, the precursor glycoprotein is gp140, cleaved into gp36 and gp105/110. Thus, in the scientific article by Vallari et al (Journal of microbiology, pages 3657-3661, 1998), there is the description of a diagnostic kit for detecting the presence of HIV-1 virus group M, HIV-1 group O and HIV-2, using three recombinant proteins derived from env regions of HIV-1 virus group M, HIV-1 group O and HIV-2. Similarly, the scientific article by Shin et al (Biochemistry and molecular biology international, volume 43, n °4, pages 713-721, 1997), describes multiantigenic peptides for detecting infections with the HIV-1 or HIV-2 virus.

However, in such tests for diagnosing several viral strains, it is necessary, during the analysis of sera by means of the ELISA (Enzyme Linked ImmunoSorbent Assay) technique, to attach to the support several recombinant proteins having different individual adsorption (or coating) characteristics, inducing problems in particular during the visualization. In addition, the multiplicity of the recombinant proteins engenders substantial production costs.

In order to avoid these problems of coating differences between the various recombinant proteins, other diagnostic tests preferentially use chimeric recombinant proteins carrying several epitopes directed against different viral proteins.

Thus, patent EP-B-0 577 894 describes the construction of a chimeric recombinant protein used for the diagnosis of AIDS. This protein carries the epitopes directed against the viral proteins derived from the gag gene of HIV-2 and against the gp120 protein of HIV-1. However, this recombinant protein does not allow the simultaneous detection of patients infected with the HIV-1 group M and O viruses, which can induce risks of false negatives (patient detected as being seronegative although he or she is carrying the virus), the consequences of which false negatives can be dramatic. In addition, this recombinant protein does not carry the epitope directed against gp41, which is nevertheless the major immunodominant epitope, which increases, here again, the risk of the appearance of a false negative. Similarly, the scientific article by Han et al (Biochemistry and molecular biology international, vol 46 n °3, 1998), describes a recombinant protein exhibiting an epitope directed against gp41 of HIV-1, and an epitope directed against gp36 of HIV-2, these two epitopes being linked via a linker peptide in order to allow accessibility to each of the epitopes. However, this recombinant protein does not allow the simultaneous detection of patients infected with the HIV-1 group M and O viruses, which can induce, here again, risks of false negatives. Patent application DE 101 06 295 describes a recombinant protein comprising several epitopes directed against HIV-1 or HIV-2, linked via linking regions, making it possible to immobilize recombinant protein on a solid support. However, the epitope regions of this recombinant protein allow recognition of antibodies directed against the products of the pol gene of the HIV virus (protease, reverse transcriptase or endonuclease) or against the sequence which constitutes the V3 loop of gp120. Since the proteins encoded by the pol gene are relatively conserved from one virus to another, antibodies directed against these proteins are not very specific. In addition, the antibodies directed against the pol antigens of a virus appear late on in infected individuals, which does not allow a diagnosis of the disease at the beginning of infection. It has also been shown that the anti-pol antigen antibody titer decreases as the disease progresses, and that, consequently, a falsely negative diagnosis could be attributed to a patient in a chronic infection phase.

As regards the sequence of the V3 loop of gp120, this sequence is hypervariable and the use of 2 or 3 “subtype-specific” sequences does not guarantee detection of all the antibodies directed against this domain: falsely negative results can therefore be obtained.

The present invention proposes to solve all the drawbacks of the state of the art by proposing a novel chimeric recombinant protein, that is easy to purify and to synthesize, and that exhibits strong immunoreactivity with respect to sera from patients liable to be infected with one or more viruses, such as HIV-1 group M and/or O or HIV-2.

The following definitions will allow a clearer understanding of the invention.
The term recombinant DNA is intended to mean a nucleotide sequence that has been artificially constructed and obtained by genetic engineering. By way of indication, said recombinant DNA can be inserted into a host organism for expression, such as in particular a bacterium, by means of an expression vector, in particular a bacterial plasmid or a bacteriophage.
The term nucleotide fragment is intended to mean a succession of at least three nucleotide acids encoding at least one amino acid.
The term cleavage site is intended to mean a site that makes it possible to separate two nucleotide fragments by the action of at least one cleavage means, such as in particular a restriction enzyme which is capable, at a cleavage site corresponding to a specific nucleotide sequence, of generating, for each strand, two ends, one having a 3′-OH group, the other a 5′-P group.
The term chimeric recombinant protein is intended to mean a protein that has been constructed artificially and obtained by genetic engineering. By way of indication, said chimeric recombinant protein can be produced by a host expression organism that has been genetically modified by insertion of the nucleotide sequence encoding said chimeric recombinant protein by means of an expression vector.
The term epitope region is intended to mean a peptide region which will interact stereospecifically with the paratopic peptide region of the antibody directed against the microorganism, such as in particular the virus, present in the serum of the patient. The most immunogenic epitope regions are referred to as immunodominant.
The term linking region is intended to mean a region providing better accessibility of the paratope regions of the various antibodies present in the serum of the patients with respect to the corresponding epitope regions of interest of the chimeric recombinant protein.
The term attaching region is intended to mean a region that makes it possible to attach said chimeric recombinant protein with respect to:
   a support, directly and/or indirectly, and/or
   a detection molecule.

The support may be made up of materials such as:
glass, a relatively inexpensive material that is inert and mechanically stable,
polymers: microtitration plates, etc.,
metals: metal chelate affinity chromatography column,
magnetic particles, as described in patent applications WO-A-97/34909, WO-A-97/45202, WO-A-98/47000 and WO-A-99/35500 filed by the applicant.

The support can then be used as an analytical support, in particular in an ELISA (Enzyme Linked ImmunoSorbent Assay), for purification steps during an affinity chromatography, for washing steps when said chimeric recombinant protein, attached to a magnetic particle, is retained by magnetization in a predetermined place.
The term detection molecule is intended to mean a molecule combined with a label for directly or indirectly generating a detectable signal. These labels can be in particular radioactive, enzymatic or fluorescent.

The attaching of the chimeric recombinant protein to said support or said detection molecule can involve ligands capable of reacting with an antiligand. By way of examples, mention may be made of the following ligand/antiligand pairs:
biotin/streptavidin,
hapten/antibody,
antigen/antibody,
peptide/antibody,
sugar/lectin.

Thus, the invention relates to a recombinant DNA encoding a chimeric recombinant protein, comprising
at least two first nucleotide fragments each encoding an epitope region of the HIV-1 virus group M or group O or of the HIV-2 virus,
at least a second nucleotide fragment encoding a linking region,
at least a third nucleotide fragment encoding an attaching region, characterized in that each first nucleotide fragment encodes at least one immunodominant region of the gp120 glycoprotein of HIV-1, of the gp41 glycoprotein of HIV-1 group M, of the gp41 glycoprotein of HIV-1 group O or of the gp36 glycoprotein of HIV-2.

It is quite evident that the variable regions of these immunodominant regions, such as in particular the V3 loop of gp120, are in no way envisioned in the present invention.

According to a preferred embodiment of the invention, said first nucleotide fragment has as its sequence any one of the sequences SEQ ID No. °3, SEQ ID No. °5, SEQ ID No. °7, SEQ ID No. °9, SEQ ID No. °27, SEQ ID No. °29 or SEQ ID No. °31.

According to another preferred embodiment of the invention, said second nucleotide fragment comprises at least one cleavage site. Preferably, said second nucleotide fragment has as its sequence at least any one of the following sequences, taken alone or in combination, SEQ ID No. °11 SEQ ID No. °13, SEQ ID No. °15, SEQ ID No. °17, SEQ ID No. °19, or SEQ ID No. °20, SEQ ID No. °33, SEQ ID No. °35, SEQ ID No. °37, SEQ ID No. °39, SEQ ID No. °41, SEQ ID No. °43 SEQ ID No. °45, or SEQ ID No. °47.

According to a preferred embodiment of the invention, said third nucleotide fragment encoding an attaching region is included in said second nucleotide fragment encoding a linking region.

According to another preferred embodiment of the invention, said third nucleotide fragment has as its sequence any one of the sequences SEQ ID No. °21, SEQ ID No. °23, SEQ ID No. °25, SEQ ID No. °33, SEQ ID No. °35, SEQ ID No. °37 or SEQ ID No. °39.

The nucleotide sequences according to the invention can be prepared by chemical synthesis and genetic engineering using techniques well known to those skilled in the art and described, for example, in Sambrook J. et al., Molecular Cloning: A Laboratory Manual, 1989.

The nucleotide sequences of the invention can be inserted into expression vectors in order to prepare the recombinant proteins of the invention.

The invention also relates to a chimeric recombinant protein encoded by a recombinant DNA, as defined above, comprising
at least two epitope regions of the HIV-1 virus group M or group O or the HIV-2 virus of at least one microorganism,
at least one linking region,
at least one attaching region.

According to a preferred embodiment of the invention, said linking region is a peptide comprising at least one glycine and/or at least one serine.

According to another preferred embodiment of the invention, said linking region has as its sequence any one of the sequences SEQ ID No. °12, SEQ ID No. °14, SEQ ID No. °16 or SEQ ID No. °18, 34, 36, 38, 40, 42, 44, 46 or 48.

According to another embodiment of the invention, said attaching region is a region rich in histidines and derivatives thereof, such as a region containing a density of histidines greater than or equal to 25%, and preferably greater than or equal to 33%.

According to another embodiment of the invention, said attaching region is a peptide comprising at least one lysine.

According to a preferred embodiment of the invention, said attaching region has as its sequence SEQ ID No. °22, 24, 26, 34, 36, 38 or 40.

The recombinant proteins of the invention can be obtained by the genetic engineering technique, which comprises the steps of:
culturing host organisms or eukaryotic cells transformed by means of a nucleotide sequence according to the invention, and
recovering the protein produced by said transformed host organisms or said transformed eukaryotic cells.

This technique is well known to those skilled in the art. For further details with regard thereto, reference may be made to the following manual: Recombinant DNA Technology I, Editors Ales Prokop, Raskesh K Bajpai; Annals of the New-York Academy of Sciences, Volume 646, 1991.

The invention also relates to an expression vector comprising a recombinant DNA as defined above.

By way of an expression vector, mention may be made, for example, of plasmids, viral vectors of the type vaccinia virus, adenovirus or baculovirus, or bacterial vectors of the type salmonella or BCG.

The expression “means required for the expression of a protein” is intended to mean any means which make it possible to obtain said protein, such as in particular a promoter, a transcription terminator, an origin of replication, and preferably a selection marker.

The vectors of the invention can also comprise sequences required for targeting the proteins to particular cellular compartments. An example of targeting may be the targeting to the endoplasmic reticulum obtained using targeting sequences such as the leader sequence derived from the adenoviral E3 protein (Ciernik I. F., et al., The Journal of Immunology, 1999, 162, 3915-3925).

By way of examples of host organisms that are suitable for the purposes of the invention, mention may be made of yeast, such as those of the following families: Saccharomyces, Schizosaccharomyces, Kluveromyces, Pichia, Hanseluna, Yarowia, Schwaniomyces and Zygosaccharomyces, Saccharomyces cerevisiae, Saccharomyces carlsbergensis and Kluveromyces lactis being preferred; and bacteria, such as E. coli and those of the following families: Lactobacillus, Lactococcus, Salmonella, Strptococcus, Bacillus and Streptomyces.

By way of eukaryotic cells, mention may be made of cells originating from animals such as mammals, reptiles, insects and equivalent. The preferred eukaryotic cells are cells originating from the Chinese hamster (CHO cells), from monkey (COS and Vero cells), from baby hamster kidney (BHK cells), from pig kidney (PK 15 cells) and from rabbit kidney (RK13 cells), human osteosarcoma cell lines (143 B cells), HeLa human cell lines and human hepatoma cell lines (of the Hep G2 cell type), and also insect cell lines (for example from Spodoptera frugiperda).

Finally, the invention relates to the use of at least one DNA as defined above and/or of at least one chimeric recombinant protein as defined above, for in vitro diagnosis. This use makes it possible to detect the HIV-1 virus group M and group O and also the HIV-2 virus.

The following examples are given by way of illustration and are in no way limiting in nature. They will make it possible to understand the invention more clearly.

### EXAMPLE 1

### Construction of the Recombinant Chimeric Proteins b-HIV72, b-HIV86 and b-HIV98 for the Recognition of Anti-HIV-1 Group O and M and -HIV-2 Antibodies

The nucleotide sequence SEQ ID No °1 was designed so as to encode a recombinant protein b-HIV72, and was cloned into an expression vector. It corresponds to the following sequence:

SEQ ID No 1:
ATG AGG GGA TCC AGA ATC CTA GCT GTG GAA AGA TAC
CTA AAG GAT CAA CAG CTC CTA GGG ATT TGG GGT TGC
TCT GGA AAA CTC ATT TGC ACC ACT GCT GTG AGC TCC
GGT TCA GGC GCT ATA GAG AAG TAC CTA CAG GAC CAG
GCG CGG CTA AAT TCA TGG GGA TGT GCG TTT AGA CAA
GTC TGC TCG AGC GGT TCT GGA GGA GGA GAT ATG AGG
GAC AAT TGG AGA AGT GAA TTA TAT AAA TAT AAA GTA
GTA AAA ATT GAA CCA TTA GGA GTA GCA CCC ACC AAG
TCT GCA GGC CGT CTG CTT GCT CTG GAA ACC CTG CTT
CAG AAC CAA CAG CTG CTT TCT CTG TGG GGT TGC AAA
GGT AAG CTG GTT TGC TAC ACC TCT GTT AAA GCT TCC
CAC CAT CAC CAT CAC CAT TGA TCT AGA

The chimeric recombinant protein b-HIV72 encoded by the sequence SEQ ID No °1 comprises 137 amino acids, for a molecular mass of 15191.5 Da. Its amino acid sequence is as follows:

SEQ ID No 2:
MRGS RILAVERYLK DQQLLGIWGC SGKLICTTAV SSGSG
AIEKYLQDQA RLNSWGCAFR QVC SSGS GGGDMRDNWR
SELYKYKVVK IEPLGVAPTK SAG RLLALETLLQ NQQLLSLWG
CKGKLVCYTS V KAS HHHHHH.

The presence of MRGS and the corresponding sequence ATG AGG GGA TCC is introduced by means of the cloning technique used in the expression vector pMR. The sequence of interest is introduced into the pMR vector between the BamHI restriction site in the 5′ position and the XbaI site in the 3′ position, which results in fusion of the MRGS sequence at the N-terminal of the protein of interest. Only the ATG initiation codon and, consequently, the Met amino acid is really essential in this sequence.

The epitope regions are indicated in bold, the attaching region in italics and the linking regions in non-bold, non-italics.

This chimeric recombinant protein b-HIV72 comprises:
a) several epitope regions (indicated in bold in SEQ ID No. 2) allowing:
   recognition of anti-HIV-1 (group M; gp41) antibodies:

The sequence SEQ ID No. 3 is derived from the HIV-1 group M viral strain (clone of reference HXB2) and corresponds to the following sequence:

SEQ ID No. 3:
AGA ATC CTA GCT GTG GAA AGA TAC CTA AAG GAT CAA
CAG CTC CTA GGG ATT TGG GGT TGC TCT GGA AAA CTC
ATT TGC ACC ACT GCT GTG.

This sequence is amplified by PCR (polymerase chain reaction) using specific amplification primers (sense primer 5′ AGT CGG ATC CAG AAT CCT AGC TGT GGA A 3′ and antisense primer 5′ GCC TGA TCC GGA GCT CAC AGC AGT GGT GCA AAT 3′); 17 PCR cycles are carried out with, in each cycle, a denaturation step at 94° C. for 1 minute (min), a hybridization step at 52° C. for 1 min and an elongation step at 72° C. for 20 seconds.

The nucleotide fragment obtained encodes the peptide corresponding to the amino acid sequence SEQ ID No. 4: RILAVERYLK DQQLLGIWGC SGKLICTTAV.
recognition of anti-HIV-1 (group O; gp41) antibodies:

The sequence SEQ ID No. 5 corresponds to an artificial DNA sequence designed based on the amino acid sequence of the HIV-1 group O viral strain [clone ANT70]. This synthetic portion was designed by selecting codons whose use is favorable to gene expression in E. coli. The sequence is as follows:

SEQ ID No. 5:
CGT CTG CTT GCT CTG GAA ACC CTG CTT CAG AAC CAA
CAG CTG CTT TCT CTG TGG GGT TGC AAA GGT AAG CTG
GTT TGC TAC ACC TCT GTT.

This sequence is constructed by PCR using 3 oligonucleotides (a sense oligonucleotide 5′ AAG TCT GCA GGC CGT CTG CTT GCT CTG GAA ACC CTG CTT CAG AAC CAA CAG CTG CTT TCT 3′ and two antisense oligonucleotides 5′ GCT ATC TAG ATC AAT GGT GAT GGT GAT GGT GGG AAG CTT TAA CAG AGG TGT AGC AAA C 3′ and 5′ AAC AGA GGT GTA GCA AAC CAG CTT ACC TTT GCA ACC CCA CAG AGA AAG CAG CTG TTG GTT 3′; 17 PCR cycles are carried out with, in each cycle, a denaturation step at 9° C. for 1 min, a hybridization step at 50° C. for 1 min and an elongation step at 68° C. for 20 seconds).

This nucleotide fragment encodes the peptide corresponding to the amino acid sequence

SEQ ID No. 6: RLLALETLLQ NQQLLSLWGC KGKLVCYTSV.

recognition of anti-HIV-2 (gp36) antibodies:

The sequence SEQ ID No. 7 is derived from the HIV-2 viral strain (clone of reference ROD) and corresponds to the following sequence:

SEQ ID No. 7:
GCT ATA GAG AAG TAC CTA CAG GAC CAG GCG CGG CTA
AAT TCA TGG GGA TGT GCG TTT AGA CAA GTC TGC.

This sequence is amplified by PCR using specific amplification primers (sense primer 5′ CTG TGA GCT CCG GTT CAG GCG CTA TAG AGA AGT ACC TA 3′ and antisense primer 5′ AGA ACC GCT CGA GCA GAC TTG TCT AAA CGC 3′; 17 PCR cycles are carried out with, in each cycle, a denaturation step at 94° C. for 1 min, a hybridization step at 52° C. for 1 min and an elongation step at 72° C. for 20 seconds).

This nucleotide fragment encodes the peptide corresponding to the amino acid sequence

SEQ ID No. 8 AIEKYLQDQA RLNSWGCAFR QVC.

recognition of anti-HIV-1 (group M: gp120) antibodies:

The sequence SEQ ID No. 9 is derived from HIV-1 group M viral strain (clone of reference HXB2) and corresponds to the following sequence:

SEQ ID No. 9:
GGA GGA GGA GAT ATG AGG GAC AAT TGG AGA AGT GAA
TTA TAT AAA TAT AAA GTA GTA AAA ATT GAA CCA TTA
GGA GTA GCA CCC ACC AAG.

This sequence is amplified by PCR using specific amplification primers (sense primer 5′ GTC TGC TCG AGC GGT TCT GGA GGA GGA GAT ATG AGG 3′ and antisense primer 5′ ACG TCC TGC AGA CTT GGT GGG TGC TAC TCC 3′; 17 PCR cycles are carried out, comprising, in each cycle, a denaturation step at 94° C. for 1 min, a hybridization step at 52° C. for 1 min and an elongation step at 72° C. for 20 seconds).

This nucleotide fragment encodes the peptide corresponding to the amino acid sequence

SEQ ID No. 10 GGGDMRDNWR SELYKYKVVK IEPLGVAPTK.

b) Linking regions, between each of the epitope regions mentioned above, allowing:
at the nucleotide level, the introduction of six sites for cleavage with restriction enzymes which may be used to modify, remove or add an epitope domain, and
at the protein level, the obtaining of flexible spacer regions that provide better accessibility of the potential antibodies to each of the domains.

Thus, the nucleotide sequence SEQ ID No. 11: G AGC TCC GGT TCA GGC makes it possible to obtain a site for cleavage with the SacI enzyme (indicated in bold), the G indicated in italics being the last base of the nucleotide sequence encoding the peptide allowing recognition of the anti-HIV-1, group M antibodies. This sequence encodes the flexible region corresponding to the peptide of sequence SEQ ID No. 12: SSG SG. The nucleotide sequence SEQ ID No. 13: C TCG AGC GGT TCT makes it possible to obtain a site for cleavage with the XhoI enzyme (indicated in bold), the C indicated in italics being the last base of the nucleotide sequence encoding the peptide allowing recognition of the anti-HIV-2 antibodies. This sequence encodes the flexible region corresponding to the peptide of sequence SEQ ID No. 14: SSGS.

The nucleotide sequence SEQ ID No. 15: TCT GCA GGC makes it possible to obtain a site for cleavage with the PstI enzyme (indicated in bold). This sequence encodes the flexible region corresponding to the peptide of sequence SEQ ID No. 16: SAG

The nucleotide sequence SEQ ID No. 17: AAA GCT TCC makes it possible to obtain a site for cleavage with the HindIII enzyme (indicated in bold). This sequence encodes the flexible region corresponding to the peptide of sequence SEQ ID No. 18: KAS.

The sequences SEQ ID No. 19: ATG AGG GGA TCC and SEQ ID No. 20: TGA TCT AGA make it possible, respectively, to obtain a site for cleavage with the BamH1 enzyme (indicated in bold) and a site for cleavage with the XbaI enzyme allowing the insertion or the extraction of the entire sequence encoding the recombinant protein according to the invention in a plasmid.

c) An attaching region allowing purification of the chimeric recombinant protein: a hexahistidine sequence is added at the C-terminal in order to subsequently facilitate the step for purifying the chimeric recombinant protein. This peptide, encoded by the nucleotide sequence SEQ ID No. °21: CAC CAT CAC CAT CAC CAT, corresponds to the sequence SEQ ID No. °22: HHHHHH.

By way of indication, this particular attaching region, comprising a succession of histidines, allows in particular the oriented attachment of the recombinant protein to a support consisting of silica or of metal oxides, as described in patent FR-B-98/04879.

The order of the sequences encoding the various immunodominant epitope regions of the chimeric recombinant protein can be optionally modified. Certain epitopes can be presented several times within the chimeric recombinant protein. The epitopes can also exhibit variations with respect to the sequences described in the example above, according to the HIV subtype or clone that they represent. The length of the linking regions can also be modified in order to improve the accessibility of an epitope. Finally, the attaching regions can be inserted into the linking regions.

Thus, the inventors have demonstrated that epitope sequences shorter than those described above can also be used. The sequences allow:
recognition of anti-HIV-1 (group M) antibodies:

The sequence SEQ ID No. °27 is derived from the HIV-1 group M viral strain (clone of reference HXB2) and corresponds to the following sequence:

SEQ ID No. °27:
GAA AGA TAC CTA AAG GAT CAA CAG CTC CTA GGG ATT
TGG GGT TGC TCT GGA AAA CTC ATT TGC ACC ACG

This nucleotide fragment encodes the peptide corresponding to the amino acid sequence

| | |
|---|---|
| SEQ ID No.°28: | ERYLKDQQLL GIWGCSGKLI CTT. |

recognition of anti-HIV-1 (group O; gp41) antibodies:

The sequence SEQ ID No. °29 corresponds to an artificial DNA sequence designed based on the amino acid sequence of the HIV-1 group O viral strain [clone ANT70]. This synthetic portion was designed by selecting codons whose use is favorable to gene expression E. coli. The sequence is as follows:

SEQ ID No.°29:
GAA ACC CTG CTT CAG AAC CAA CAG CTG CTT TCT CTG
TGG GGT TGC AAA GGT AAG CTG GTT TGC TAG ACC.

This nucleotide fragment encodes the peptide corresponding to the amino acid sequence

| | |
|---|---|
| SEQ ID No.°30: | ETLLQNQQLL SLWGCKGKLV CYT. |

recognition of anti-HIV-2 (gp36) antibodies:

The sequence SEQ ID No. °31 is derived from the HIV-2 viral strain (clone of reference ROD) and corresponds to the following sequence:

SEQ ID No.°31:
CTA AAT TCA TGG GGA TGT GCG TTT AGA CAA GTC
TGC.

This nucleotide fragment encodes the peptide corresponding to the amino acid sequence

| | |
|---|---|
| SEQ ID No.°32: | LNSWGCAFR QVC. |

In addition, the inventors also used the following linking regions:
the nucleotide sequence SEQ ID No. °41 CTG CAC CAT ATC CTG GAA GCC CAG CGT ATG GAA TGG CAC CCG CAC AAA GGT TCT GGA TCC which corresponds to the amino acid sequence SEQ ID No. °42 LHHILEAQRM EWHPHKGSGS;
the nucleotide sequence SEQ ID No. °43 CTG CAC CAT ATC CTG GAG GCT CAA CGT ATG GAG TGG CGC GAA TCC CAT GGT which corresponds to the amino acid sequence SEQ ID No. °44 LHHILEAQRM EWRESHG;
the nucleotide sequence SEQ ID No. °45 GGT CTG AAC GAC ATC CTG GAA GCC CAG CGT ATG GAA TGG CAC GAG TCT GCA GGC which corresponds to the amino acid sequence SEQ ID No. °46 GLKDILEAQR MEWHESAG;
the nucleotide sequence SEQ ID No. °47 CTG AAC GAT ATT TTC GAA GCG CAG CGT ATT GAA TGG CAT GAG GGT TCT GGA TCC which corresponds to the amino acid sequence SEQ ID No. °48 LNDIFEAQRI EWHEGSGS.

The replacement of an arginine with a lysine within a linking region as defined above makes it possible to attach a biotin, which makes it possible to obtain an attaching region within the linking region.

The inventors thus used the following sequences which made it possible not only to link the epitope regions to one another, but also to attach the chimeric recombinant protein according to the invention to a support, or to facilitate its purification.

Thus, the inventors also used the following linking regions:
the nucleotide sequence SEQ ID No. °33 CTG CAC CAT ATC CTG GAA GCC CAG AAA ATG GAA TGG CAC CCG CAC AAA GGT TCT GGA TCC which corresponds to the amino acid sequence SEQ ID No. °34 LHHILEAQKM EWHPHKGSGS;
the nucleotide sequence SEQ ID No. °35 CTG CAC CAT ATC CTG GAG GCT CAA AAG ATG GAG TGG CGC GAA TCC CAT GGT TCC CAT GGT which corresponds to the amino acid sequence SEQ ID No. °36 LHHILEAQKM EWRESHG;
the nucleotide sequence SEQ ID No. °37 GGT CTG AAC GAC ATC CTG GAA GCC CAG AAA ATG GAA TGG CAC GAG TCT GCA GGC which corresponds to the sequence SEQ ID No. °38 GLKDILEAQK MEWHESAG;
the nucleotide sequence SEQ ID No. °39 CTG AAC GAT ATT TTC GAA GCG CAG AAG ATT GAA TGG CAT GAG GGT TCT GGA TCC which corresponds to the amino acid sequence SEQ ID No. °40 LNDIFEAQKI EWHEGSGS.

The sequences presented above made it possible to construct the chimeric recombinant protein bHIV86.

Thus, the nucleotide sequence SEQ ID No. °49 was designed so as to encode a recombinant protein according to the invention, and was cloned into an expression vector.

SEQ ID No.°49:
ATG AGG GGA TCT CTG CAC CAT ATC CTG GAA GCC CAG
AAA ATG GAA TGG CAC CCG CAC AAA GGT TCT GGA TCC
GAA AGA TAC CTA AAG GAT CAA CAG CTC CTA GGG ATT
TGG GGT TGC TCT GGA AAA CTC ATT TGC ACC ACG AGC
TCC CTG CAC CAT ATC CTG GAG GCT CAA AAG ATG GAG
TGG CGC GAA TCC CAT GGT CTA AAT TCA TGG GGA TGT
GCG TTT AGA CAA GTC TGC TCG AGC GGT CTG AAG GAC
ATC CTG GAA GCC CAG AAA ATG GAA TGG CAC GAG TCT
GCA GGC GAA ACC CTG CTT CAG AAC CAA CAG CTG CTT
TCT CTG TGG GGT TGC AAA GGT AAG CTG GTT TGC TAC
ACC AAA GCT TCC CAC CAT CAC CAT CAC CAT TGA TCT
AGA.

The chimeric recombinant protein encoded by the sequence SEQ ID No. °50 is as follows:

SEQ ID No.°50:

MRGSLHHILE AQKMEWHPHK GSGSERYLKD QQLLGIWGCS
GKLICTTSSL HHILEAQKME WRESHGLNSW GCAFRQVCSS
GLKDILEAQK MEWHESAGET LLQNQQLLSL WGCKGKLVCY
TKAS [symbol] .

The epitope regions are indicated in bold, the attaching region in italics and the linking regions in non-bold, non-italics.

Similarly, the sequences presented above made it possible to construct the chimeric recombinant protein bHIV98 in which the hexahistidine sequence has been shifted to the N-terminal in order to facilitate the purification of said protein.

Thus, the nucleotide sequence SEQ ID No. °51 was designed so as to encode a recombinant protein according to the invention, and was cloned into an expression vector.

SEQ ID No.°51:
ATG AGG GGA TCT CAC CAT CAC CAT CAC CAT GGT CTG
AAC GAT ATT TTC GAA GCG CAG AAG ATT GAA TGG CAT
GAG GGT TCT GGA TCC GAA AGA TAC CTA AAG GAT CAA
CAG CTC CTA GGG ATT TGG GGT TGC TCT GGA AAA CTC
ATT TGC ACC ACG AGC TCC CTG CAC CAT ATC CTG GAG
GCT CAA AAG ATG GAG TGG CGC GAA TCC CAT GGT CTA
AAT TCA TGG GGA TGT GCG TTT AGA CAA GTC TGC TCG
AGC GGT CTG AAG GAC ATC CTG GAA GCC CAG AAA ATG
GAA TGG CAC GAG TCT GCA GGC GAA ACC CTG CTT CAG
AAC CAA CAG CTG CTT TCT CTG TGG GGT TGC AAA GGT
AAG CTG GTT TGC TAC ACC TGA A GCT T.

The chimeric recombinant protein encoded by the sequence SEQ ID No. °52 is as follows:

SEQ ID No.°52:

MRGSHHHHHH GLNDIFEAQK IEWHEGSGSE RYLKDQQLLG
IWGCSGKLIC TTSSLHHILE AQKMEWRESH GLNSWGCAFR
QVCSSGLKDI LEAQKMEWHE SAGETLLQNQ QLLSLWGCKG
KLVCYT.

The epitope regions are indicated in bold, the attaching region in italics and the linking regions in non-bold, non-italics.

### EXAMPLE 2

### Expression and Purification of the Chimeric Recombinant Proteins b-HIV 72, b-HIV86 and b-HIV98 of Example 1

The first step consists in inserting the sequence SEQ ID No. °1 (Example 1) into an expression vector (pMR) and then in transforming an E. coli bacterium (strain BL21) with the plasmid construct obtained according to a conventional cloning protocol known to those skilled in the art. The transformed bacteria are selected by means of their ampicillin resistance carried by the pMR vector.

One recombinant bacterial clone is then selected in order to seed a preculture of 40 ml of 2×YT medium (16 g/l tryptone; 10 g/l yeast extract; 5 g/l NaCl, pH 7.0) containing 100 μg/ml of ampicillin. After incubation for 15 to 18 h at 37° C. with shaking at 250 rpm, this preculture is used to seed 1 liter of 2×YT medium containing 2% glucose and 100 μg/ml of ampicillin. This culture is incubated at 37° C. with shaking at 250 rpm. When the OD_{600 nm} has reached 0.7-0.9, IPTG (isopropyl-β-D-thiogalactoside, Eurogentec) is added to the culture medium at a concentration of 0.5 mM, and the culture is continued for 4 h. The IPTG makes it possible to induce the expression of the recombinant chimeric protein SEQ ID No. °2, No. °50 or No. °52, which accumulates in the bacteria in the form of inclusion bodies. After induction for 4 h, the culture is centrifuged at 6000 rpm for 30 min at 4° C. and the bacterial pellet is frozen at −80° C. In order to extract the recombinant protein from the inclusion bodies, the thawed bacteria are lysed. For this, the bacterial pellets corresponding to a culture of one liter are taken up in 100 ml of lysis buffer (1×PBS containing protease inhibitors: lysozyme: 1 mg/ml; benzonase: 2.5 units per ml (Novagen®) and Mg²⁺: 1 mM) by vortexing until a homogeneous suspension is obtained. This solution is incubated at ambient temperature for 1 hour with shaking. The solution is then centrifuged for 30 min at 4° C. at 10 000 g.

The pellet obtained contains the inclusion bodies. This pellet is suspended in 50 ml of solubilizing buffer (sodium bicarbonate: 40 mM; NaCl: 300 mM; SDS: 1%; β-mercaptoethanol: 20 mM, pH 9.6) containing protease inhibitors (complete EDTA-free, Roche®). The solution thus obtained is incubated for 16 to 18 h with stirring, at between 18 and 25° C. It is then diluted one-in-four with a 2×PBS buffer containing 8 mM of imidazole and protease inhibitors (complete EDTA-free, Roche®) at pH 8.0. Centrifugation at 10 000 g for 30 min at 20° C. makes it possible to obtain a clear supernatant, which is filtered through a 0.45μ filter and purified by affinity chromatography on a metal chelate column (nickel-nitrilotriacetic acid matrix (Ni-NTA, Qiagen)). The 200 ml of sample are loaded (1 m/min) at 18-25° C. onto an 8 ml Ni-NTA gel column equilibrated in A1 buffer (2×PBS, 4 M urea, 6 mM imidazole, pH 7.8 containing 5 mM β-mercaptoethanol) or A2 buffer (2×PBS, 0.25% SDS, 6 mM imidazole, pH 7.8 containing 5 mM β-mercaptoethanol). The column is then washed in A1 or A2 buffer, until an OD_{280 nm}=0 is obtained at the column outlet. Elution of the recombinant protein is obtained by application of a buffer B1 (2×PBS, 4M urea, 100 mM imidazole, pH 7.5, containing 5 mM β-mercaptoethanol) or B2 (2×PBS, 0.25% SDS, 100 mM imidazole, pH 7.5, containing 5 mM β-mercaptoethanol). Amounts of the order of 50 mg of purified recombinant protein can be obtained from one liter of culture.

The recombinant protein thus purified is subjected to a denaturing treatment by means of the addition of SDS (1500 molecules per molecule of recombinant protein), 5 mM DTT, 50 mM sodium bicarbonate at pH 9.6, and heating at 37° C. for 30 min. The SDS molecules/recombinant protein molecules stoichiometry can be modified (ideally decreased if the heating time or temperature is increased). For example, similar results are obtained by adding 250 molecules of SDS/molecule of recombinant protein, 5 mM DTT, 50 mM sodium bicarbonate at pH 9.6, and heating at 40° C. for 2 hours.

The protein thus denatured is stabilized by adding polyethylene glycol (MW 3350, in particular) for a stoichiometry of 10 molecules of PEG per molecule of protein, and then dialyzed at 4° C. for 18 to 24 hours against a 50 mM sodium bicarbonate buffer containing 1 mM EDTA, 0.01% SDS and 1 mg/l PEG, pH 9.6.

The expression and the purification of the chimeric recombinant proteins b-HIV86 and b-HIV98 were carried out in a comparable manner, with the exception of the step of denaturation with SDS and DTT, which is not necessary during the purification of the b-HIV86 and b-HIV98 proteins. The use of beta mercaptoethanol in the purification buffers is not necessary either.

### EXAMPLE 3

### Evaluation and Validation of the Chimeric Recombinant Proteins b-HIV 72, b-HIV 86 and b-HIV 98 in a VIDAS® Test (bioMérieux)

This validation is carried out by means of a VIDAS® test using a solution of recombinant chimeric protein obtained according to Examples 1 and 2 and having undergone the denaturing treatment described in Example 2.

The principle of the VIDAS® test is as follows: a pipette tip device constitutes the solid support which also serves as a pipetting system for the reagents present in the strip. The recombinant protein is attached to the pipette tip device. After a dilution step, the sample is suctioned back and forth several times inside the pipette tip device. This allows the anti-HIV IgGs of the sample to bind to the recombinant protein. The unbound components are removed by washing. An alkaline phosphatase (ALP)-conjugated anti-human IgG antibody is then incubated in the pipette tip device, where it binds to the anti-HIV IgGs. Washing steps remove the unbound conjugate.

During the final visualizing step, the ALP substrate, 4-methylumbelliferyl phosphate, is hydrolyzed to 4-methylumbelliferone, the emitted fluorescence of which at 450 nm is measured. The intensity of the fluorescence is measured by means of the Vidas® optical system and is proportional to the presence of anti-HIV IgGs present in the sample. The results are analyzed automatically by the VIDAS® and expressed as RFV (Relative Fluorescent Value).

In this example, a solution of recombinant protein obtained according to Examples 1 and 2 (1.2 μg in one milliliter of 50 mM sodium bicarbonate buffer containing 0.01% SDS, pH 9.6-9.8) is incubated with the VIDAS® pipette tip devices for 18 to 24 h at ambient temperature (120 μl/pipette tip device). The pipette tip devices are then incubated in a passivation buffer (330 μl/pipette tip device of Duo HIV buffer containing 3% of calf serum) for 18 to 24 h at ambient temperature.

Test solutions (Etablissement Francais du Sang, France [French Blood Bank]), of known HIV serology (28 μl of serum, of known HIV status, diluted in 300 μl of 1×PBS buffer containing 8.76 g/l NaCl, 2.5% (v/v) tween 20, 2.5 g/l powdered skimmed milk, 20 g/l albumin and 3% (v/v) calf serum, pH 6.1) are then brought into contact with the pipette tip devices exhibiting the recombinant proteins of Example 1, for 13 min and 20 seconds (80 cycles of pipetting/reverse flow of 10 seconds). A washing step is then carried out in buffer containing 24.23 g/l Tris, 23.22 g/l maleic acid, 0.05% (v/v) tween 20, 6 g/l NaOH and 8.77 g/l NaCl, pH 6.1. An ALP-conjugated anti-human Fc antibody solution (P5F2F7) is diluted to 1/5000 and incubated in contact with the pipette tip device for 5 min (with 30 cycles of pipetting/reverse flow of 10 seconds each). A final washing step is carried out in Duo HIV buffer, before the final visualization step.

The results obtained are expressed in RFV (Relative Fluorescent Value). The RFV values greater than or equal to 250 are arbitrarily considered as coming from an HIV-seropositive serum. The lower values are negative. The results obtained with the recombinant protein obtained according to Examples 1 and 2 are all in agreement with the HIV serology, determined beforehand by means of the VIDAS HIV Duo® test (bioMérieux®).

The use of such a recombinant protein in a VIDAS® test clearly makes it possible to determine the HIV serology of the samples.

TABLE 1
Experimental validation of the recombinant protein
b-HIV72 obtained according to Examples 1 and 2

| | Serum | RFV (relative |
|---|---|---|
| Calibrating solution/sera | References | fluorescent value) |
| HIV-1-M-positive sera | 9991574 | 9820 |
| | 9991504 | 9346 |
| | 9991544 | 9621 |
| | 9991524 | 9735 |
| | 9991500 | 9914 |
| HIV-1-O-positive sera | 48 | 786 |
| HIV-2-positive sera | 7312A | 9174 |
| | JS8-1002 0008 | 8475 |
| | JS8-1002 0009 | 9532 |
| | JS8-1002 0010 | 9472 |
| | JS8-1002 0013 | 9526 |
| | JS8-1002 0014 | 9695 |
| | JS8-1002 0016 | 9872 |
| | JS8-1002 0017 | 9090 |
| | JS8-1002 0018 | 9241 |
| | JS8-1002 0020 | 9391 |
| HIV-seronegative sera | 203 | 139 |
| | 222 | 171 |
| | 262 | 105 |
| | 263 | 84 |
| | 280 | 182 |
| | 285 | 86 |
| | 287 | 127 |
| | 291 | 173 |
| | g10653 | 193 |
| | g12290 | 122 |
| | g13461 | 170 |
| | g13818 | 192 |
| | g13830 | 106 |
| | p03512 | 176 |

Comparable results were obtained with the chimeric recombinant proteins b-HIV86 and b-HIV98.

### EXAMPLE 4

### Construction of a Biotinylated Recombinant Protein

A consensus sequence for biotinylation in vivo in E. coli as described by Schatz, (Bio/technology, vol. 11, 1993) can be fused to SEQ ID No. 2.

Addition of the sequence SEQ ID No. °23: 5′ CTG CAC CAT ATC CTG GAA GCC CAG AAA ATG GAA TGG CAC CCG CAC, encoding the peptide of sequence SEQ ID No. °24: LHHILEAQKM EWHPH, makes it possible to biotinylate the recombinant protein b-HIV72 obtained according to Examples 1 and 2 in order to use an avidine protein conjugated to an enzyme for the visualizing phase in an EIA assay.

The expression and purification conditions described in Example 2 remain valid with a few modifications. The bacteria transformed with the recombinant plasmid may be of the BL21 or AVB101 type (Avidity, LLC). The culture medium used for the expression may be of the type: 2×YT (16 g/l tryptone; 10 g/l yeast extract; 5 g/l NaCl, pH 7.0) containing 100 μg/ml of ampicillin and supplemented with 12 μg per ml of biotin.

### EXAMPLE 5

### Evaluation and Validation of the Recombinant Chimeric Protein Biotinylated in vivo as Described in Example 4 in a VIDAS® Sandwich Test

This validation is according to a VIDAS® protocol described in Example 3 and modified as follows: the non-biotinylated bHIV-72 recombinant chimeric protein obtained according to Example 1 is attached to the solid phase (VIDAS® pipette tip device) as described in Example 3. After incubation of the diluted sample with the pipette tip device and then washing, the anti-HIV IgGs bound to the pipette tip devices are incubated with a recombinant chimeric protein biotinylated in vivo and obtained according to Example 4. After washing, the biotinylated recombinant proteins attached to the pipette tip device react with a solution of ALP-conjugated streptavidin. The final visualizing step is in accordance with the description of Example 3.

The results are given in Table 2. The RFV values greater than or equal to 250 are arbitrarily considered to be positive. The use of such a recombinant protein in a VIDAS® test clearly makes it possible to determine the HIV serology of the samples.

TABLE 2
Experimental validation of the recombinant protein
obtained according to Examples 4 to 5

| Calibrating solution/sera | Serum references | RFV |
|---|---|---|
| HIV-negative sera | CTS 8 | 195 |
| | g06976 | 214 |
| | g31377 | 170 |
| HIV 1-M-positive sera | 9991574 | 4708 |
| | 9991504 | 1395 |
| HIV-2-positive sera | JS8-1002 0008 | 2562 |
| | JS8-1002 0009 | 3754 |

### EXAMPLE 6

### Evaluation and Validation of the Chimeric Recombinant Proteins b-HIV-86 and b-HIV-98 in a VIDAS® Sandwich Test

The presence of the sequence SEQ ID No. °33, 35 37 or 39 encoding, respectively, the peptides SEQ ID Nos. °34, 36, 38 and 40 allows the b-HIV86 and b-HIV98 proteins to be biotinylated in vivo, in a manner comparable to that which is described in Example 4.

This validation is according to a VIDAS® protocol described in Example 3 and modified as follows: the non-biotinylated recombinant chimeric protein bHIV-72 obtained according to Example 1 is attached to the solid phase (VIDAS® pipette tip device) as described in Example 3. After incubation of the diluted sample with the pipette tip device and then washing, the anti-HIV IgGs bound to the pipette tip devices are incubated with a recombinant chimeric protein biotinylated in vivo (bHIV-86 or bHIV-98). After washing, the biotinylated recombinant proteins attached to the pipette tip device react with a solution of ALP-conjugated streptavidin. The final visualizing step is in accordance with the description of Example 3.

Results comparable to those described in Example 5 were obtained with the bHIV-86 and bHIV-98 proteins.

### EXAMPLE 7

### Improvement in the Sensitivity of the Chimeric Recombinant Protein

In order to further increase the sensitivity of anti-HIV antibody recognition of the recombinant protein obtained according to the invention, new epitopes characteristic of certain HIV subtypes can be added, or one of the epitopes described can be duplicated in the sequence of the chimeric protein.

### EXAMPLE 8

### Addition of a Hexalysine Sequence to the Recombinant Protein in Order to Facilitate the Coupling of an Enzyme or a Biotin, in Particular on the β-amino Function of the Lysine

A sequence encoding six lysines can be fused in the 3′position of SEQ ID No. 2. The addition of the DNA sequence for SEQ ID No. 25: AAG AAA AAG AAA AAG AAA, encoding the peptide of sequence SEQ ID No. 26: KKKKKK, allows the oriented coupling of an enzyme or biotin at the C-terminal of the chimeric protein. The coupling of this recombinant protein to alkaline phosphatase in particular makes it possible to use the coupled protein in a sandwich format for detecting anti-HIV antibodies.

1. A recombinant DNA encoding a chimeric recombinant protein, comprising
at least two first nucleotide fragments each encoding an epitope region of the HIV-1 virus group M or group O or of the HIV-2 virus,
at least a second nucleotide fragment encoding a linking region,
at least a third nucleotide fragment encoding an attaching region, characterized in that each first nucleotide fragment encodes at least one immunodominant region of the gp120 glycoprotein of HIV-1, of the gp41 glycoprotein of HIV-1 group M, of the gp41 glycoprotein of HIV-1 group O or of the gp36 glycoprotein of HIV-2.

2. The DNA as claimed in claim 1, characterized in that said first nucleotide fragment has as its sequence any one of the sequences SEQ ID No. °3, SEQ ID No. °5, SEQ ID No. °7, SEQ ID No. °9, SEQ ID No. °27, SEQ ID No. °29 or SEQ ID No. °31.

3. The recombinant DNA as claimed in claim 1, characterized in that said second nucleotide fragment comprises at least one cleavage site.

4. The recombinant DNA as claimed in claim 1, characterized in that said second nucleotide fragment has as its sequence at least any one of the following sequences, taken alone or in combination, SEQ ID No. °11 SEQ ID No. °13, SEQ ID No. °15, SEQ ID No. °17, SEQ ID No. °9, ou SEQ ID No. °20, SEQ ID No. °33, SEQ ID No. °35, SEQ ID No. °37, SEQ ID No. °39, SEQ ID No. °41, SEQ ID No. °43 SEQ ID No. °45, or SEQ ID No. °47

5. The recombinant DNA as claimed in claim 1, characterized in that said third nucleotide fragment encoding an attaching region is included in said second nucleotide fragment encoding a linking region.

6. The DNA as claimed in claim 1, characterized in that said third nucleotide fragment has as its sequence any one of the sequences SEQ ID No. °21, SEQ ID No. °23, SEQ ID No. °25, SEQ ID No. °33, SEQ ID No. °35, SEQ ID No. °37 or SEQ ID No. °39.

7. A chimeric recombinant protein encoded by a recombinant DNA as claimed in claim 1, comprising
at least two epitope regions of the HIV-1 virus group M or group O or the HIV-2 virus,
at least one linking region,
at least one attaching region.

8. The protein as claimed in claim 7, characterized in that said linking region is a peptide comprising at least one glycine and/or at least one serine.

9. The protein as claimed in claim 8, characterized in that said linking region has as its sequence any one of the sequences SEQ ID No. °12, SEQ ID No. °14, SEQ ID No. °16 or SEQ ID No. °18, 34, 36, 38, 40, 42, 44, 46 or 48.

10. The protein as claimed in claim 7, characterized in that said attaching region is a region rich in histidines and derivatives thereof, such as a region containing a density of histidines greater than or equal to 25%, and preferably greater than or equal to 33%.

11. The protein as claimed in claim 7, characterized in that said attaching region is a peptide comprising at least one lysine.

12. The protein as claimed in claim 7, characterized in that said attaching region has as its sequence SEQ ID No. °22, 24, 26, 34, 36, 38 or 40.

13. An expression vector comprising a recombinant DNA as claimed in claim 1.

14. Canceled.


SEQUENCE LISTING (61 sequences)

SEQ ID NO: 1 - DNA, 423 bp, Artificial Sequence
  DNA encoding a recombinant protein b-HIV72
atgaggggatccagaatcctagctgtggaaagatacctaaaggatcaacagctcctagggatttggggttgctctggaaaactcatttgcaccactgctgtgagctccggttcaggcgctatagagaagtacctacaggaccaggcgcggctaaattcatggggatgtgcgtttagacaagtctgctcgagcggttctggaggaggagatatgagggacaattggagaagtgaattatataaatataaagtagtaaaaattgaaccattaggagtagcacccaccaagtctgcaggccgtctgcttgctctggaaaccctgcttcagaaccaacagctgctttctctgtggggttgcaaaggtaagctggtttgctacacctctgttaaagcttcccaccatcaccatcaccattgatctaga

SEQ ID NO: 2 - protein, 138 aa, Artificial Sequence
  chimeric recombinant protein b-HIV72
MRGSRILAVERYLKDQQLLGIWGCSGKLICTTAVSSGSGAIEKYLQDQARLNSWGCAFRQVCSSGSGGGDMRDNWRSELYKYKVVKIEPLGVAPTKSAGRLLALETLLQNQQLLSLWGCKGKLVCYTSVKASHHHHHH

SEQ ID NO: 3 - DNA, 90 bp, Human immunodeficiency virus type 1
agaatcctagctgtggaaagatacctaaaggatcaacagctcctagggatttggggttgctctggaaaactcatttgcaccactgctgtg

SEQ ID NO: 4 - protein, 30 aa, Human immunodeficiency virus type 1
RILAVERYLKDQQLLGIWGCSGKLICTTAV

SEQ ID NO: 5 - DNA, 90 bp, Human immunodeficiency virus type 1
cgtctgcttgctctggaaaccctgcttcagaaccaacagctgctttctctgtggggttgcaaaggtaagctggtttgctacacctctgtt

SEQ ID NO: 6 - protein, 30 aa, Human immunodeficiency virus type 1
RLLALETLLQNQQLLSLWGCKGKLVCYTSV

SEQ ID NO: 7 - DNA, 69 bp, Human immunodeficiency virus type 2
gctatagagaagtacctacaggaccaggcgcggctaaattcatggggatgtgcgtttagacaagtctgc

SEQ ID NO: 8 - protein, 23 aa, Human immunodeficiency virus type 2
AIEKYLQDQARLNSWGCAFRQVC

SEQ ID NO: 9 - DNA, 90 bp, Human immunodeficiency virus type 1
ggaggaggagatatgagggacaattggagaagtgaattatataaatataaagtagtaaaaattgaaccattaggagtagcacccaccaag

SEQ ID NO: 10 - protein, 30 aa, Human immunodeficiency virus type 1
GGGDMRDNWRSELYKYKVVKIEPLGVAPTK

SEQ ID NO: 11 - DNA, 16 bp, Artificial Sequence
  DNA sequence corresponding to SEQ ID NO 12
gagctccggttcaggc

SEQ ID NO: 12 - protein, 5 aa, Artificial Sequence
  linking region
SSGSG

SEQ ID NO: 13 - DNA, 13 bp, Artificial Sequence
  DNA sequence corresponding to SEQ ID NO 14
ctcgagcggttct

SEQ ID NO: 14 - protein, 4 aa, Artificial Sequence
  linking region
SSGS

SEQ ID NO: 15 - DNA, 9 bp, Artificial Sequence
  DNA sequence corresponding to SEQ ID NO 16
tctgcaggc

SEQ ID NO: 16 - protein, 3 aa, Artificial Sequence
  linking region
SAG

SEQ ID NO: 17 - DNA, 9 bp, Artificial Sequence
  DNA sequence corresponding to SEQ ID NO 18
aaagcttcc

SEQ ID NO: 18 - protein, 3 aa, Artificial Sequence
  linking region
KAS

SEQ ID NO: 19 - DNA, 12 bp, Artificial Sequence
  DNA sequence corresponding to a linking region
atgaggggatcc

SEQ ID NO: 20 - DNA, 17 bp, Artificial Sequence
  DNA sequence corresponding to a linking region
artcaatgaggggatcc

SEQ ID NO: 21 - DNA, 18 bp, Artificial Sequence
  DNA sequence encoding SEQ ID NO. 22
caccatcaccatcaccat

SEQ ID NO: 22 - protein, 6 aa, Artificial Sequence
  attaching region
HHHHHH

SEQ ID NO: 23 - DNA, 45 bp, Artificial Sequence
  DNA sequence corresponding to SEQ ID NO 24
ctgcaccatatcctggaagcccagaaaatggaatggcacccgcac

SEQ ID NO: 24 - protein, 15 aa, Artificial Sequence
  biotinylated recombinant protein
LHHILEAQKMEWHPH

SEQ ID NO: 25 - DNA, 18 bp, Artificial Sequence
  DNA sequence corresponding to SEQ ID NO 26
aagaaaaagaaaaagaaa

SEQ ID NO: 26 - protein, 6 aa, Artificial Sequence
  coupling sequence
KKKKKK

SEQ ID NO: 27 - DNA, 69 bp, Human immunodeficiency virus type 1
gaaagatacctaaaggatcaacagctcctagggatttggggttgctctggaaaactcatttgcaccacg

SEQ ID NO: 28 - protein, 23 aa, Human immunodeficiency virus type 1
ERYLKDQQLLGIWGCSGKLICTT

SEQ ID NO: 29 - DNA, 69 bp, Human immunodeficiency virus type 1
gaaaccctgcttcagaaccaacagctgctttctctgtggggttgcaaaggtaagctggtttgctacacc

SEQ ID NO: 30 - protein, 23 aa, Human immunodeficiency virus type 1
ETLLQNQQLLSLWGCKGKLVCYT

SEQ ID NO: 31 - DNA, 36 bp, Human immunodeficiency virus type 2
ctaaattcatggggatgtgcgtttagacaagtctgc

SEQ ID NO: 32 - protein, 12 aa, Human immunodeficiency virus type 2
LNSWGCAFRQVC

SEQ ID NO: 33 - DNA, 60 bp, Artificial Sequence
  DNA sequence corresponding to SEQ ID NO 34
ctgcaccatatcctggaagcccagaaaatggaatggcacccgcacaaaggttctggatcc

SEQ ID NO: 34 - protein, 20 aa, Artificial Sequence
  linking region
LHHILEAQKMEWHPHKGSGS

SEQ ID NO: 35 - DNA, 51 bp, Artificial Sequence
  DNA sequence corresponding to SEQ ID NO 36
ctgcaccatatcctggaggctcaaaagatggagtggcgcgaatcccatggt

SEQ ID NO: 36 - protein, 17 aa, Artificial Sequence
  linking region
LHHILEAQKMEWRESHG

SEQ ID NO: 37 - DNA, 54 bp, Artificial Sequence
  DNA sequence encoding SEQ ID NO 38
ggtctgaaggacatcctggaagcccagaaaatggaatggcacgagtctgcaggc

SEQ ID NO: 38 - protein, 18 aa, Artificial Sequence
  linking region
GLKDILEAQKMEWHESAG

SEQ ID NO: 39 - DNA, 54 bp, Artificial Sequence
  DNA sequence corresponding to SEQ ID NO 40
ctgaacgatattttcgaagcgcagaagattgaatggcatgagggttctggatcc

SEQ ID NO: 40 - protein, 18 aa, Artificial Sequence
  linking region
LNDIFEAQKIEWHEGSGS

SEQ ID NO: 41 - DNA, 60 bp, Artificial Sequence
  DNA sequence corresponding to SEQ ID NO 42
ctgcaccatatcctggaagcccagcgtatggaatggcacccgcacaaaggttctggatcc

SEQ ID NO: 42 - protein, 20 aa, Artificial Sequence
  linking region
LHHILEAQRMEWHPHKGSGS

SEQ ID NO: 43 - DNA, 51 bp, Artificial Sequence
  DNA sequence corresponding to SEQ ID NO 44
ctgcaccatatcctggaggctcaacgtatggagtggcgcgaatcccatggt

SEQ ID NO: 44 - protein, 17 aa, Artificial Sequence
  linking region
LHHILEAQRMEWRESHG

SEQ ID NO: 45 - DNA, 54 bp, Artificial Sequence
  DNA sequence corresponding to SEQ ID NO 46
ggtctgaaggacatcctggaagcccagcgtatggaatggcacgagtctgcaggc

SEQ ID NO: 46 - protein, 18 aa, Artificial Sequence
  linking region
GLKDILEAQRMEWHESAG

SEQ ID NO: 47 - DNA, 54 bp, Artificial Sequence
  DNA sequence corresponding to SEQ ID NO 48
ctgaacgatattttcgaagcgcagcgtattgaatggcatgagggttctggatcc

SEQ ID NO: 48 - protein, 18 aa, Artificial Sequence
  linking region
LNDIFEAQRIEWHEGSGS

SEQ ID NO: 49 - DNA, 399 bp, Artificial Sequence
  DNA sequence corresponding to a recombinant protein
atgaggggatctctgcaccatatcctggaagcccagaaaatggaatggcacccgcacaaaggttctggatccgaaagatacctaaaggatcaacagctcctagggatttggggttgctctggaaaactcatttgcaccacgagctccctgcaccatatcctggaggctcaaaagatggagtggcgcgaatcccatggtctaaattcatggggatgtgcgtttagacaagtctgctcgagcggtctgaaggacatcctggaagcccagaaaatggaatggcacgagtctgcaggcgaaaccctgcttcagaaccaacagctgctttctctgtggggttgcaaaggtaagctggtttgctacaccaaagcttcccaccatcaccatcaccattgatctaga

SEQ ID NO: 50 - protein, 130 aa, Artificial Sequence
  chimeric recombinant protein
MRGSLHHILEAQKMEWHPHKGSGSERYLKDQQLLGIWGCSGKLICTTSSLHHILEAQKMEWRESHGLNSWGCAFRQVCSSGLKDILEAQKMEWHESAGETLLQNQQLLSLWGCKGKLVCYTKASHHHHHH

SEQ ID NO: 51 - DNA, 386 bp, Artificial Sequence
  DNA sequence corresponding to recombinant protein
atgaggggatctcaccatcaccatcaccatggtctgaacgatattttcgaagcgcagaagattgaatggcatgagggttctggatccgaaagatacctaaaggatcaacagctcctagggatttggggttgctctggaaaactcatttgcaccacgagctccctgcaccatatcctggaggctcaaaagatggagtggcgcgaatcccatggtctaaattcatggggatgtgcgtttagacaagtctgctcgagcggtctgaaggacatcctggaagcccagaaaatggaatggcacgagtctgcaggcgaaaccctgcttcagaaccaacagctgctttctctgtggggttgcaaaggtaagctggtttgctacacctgaagctt

SEQ ID NO: 52 - protein, 126 aa, Artificial Sequence
  chimeric recombinant protein
MRGSHHHHHHGLNDIFEAQKIEWHEGSGSERYLKDQQLLGIWGCSGKLICTTSSLHHILEAQKMEWRESHGLNSWGCAFRQVCSSGLKDILEAQKMEWHESAGETLLQNQQLLSLWGCKGKLVCYT

SEQ ID NO: 53 - DNA, 28 bp, Artificial Sequence
  sense primer
agtcggatccagaatcctagctgtggaa

SEQ ID NO: 54 - DNA, 33 bp, Artificial Sequence
  antisense primer
gcctgatccggagctcacagcagtggtgcaaat

SEQ ID NO: 55 - DNA, 60 bp, Artificial Sequence
  sense primer
aagtctgcaggccgtctgcttgctctggaaaccctgcttcagaaccaacagctgctttct

SEQ ID NO: 56 - DNA, 58 bp, Artificial Sequence
  antisense primer
gctatctagatcaatggtgatggtgatggtgggaagctttaacagaggtgtagcaaac

SEQ ID NO: 57 - DNA, 60 bp, Artificial Sequence
  antisense primer
aacagaggtgtagcaaaccagcttacctttgcaaccccacagagaaagcagctgttggtt

SEQ ID NO: 58 - DNA, 38 bp, Artificial Sequence
  sense primer
ctgtgagctccggttcaggcgctatagagaagtaccta

SEQ ID NO: 59 - DNA, 30 bp, Artificial Sequence
  antisense primer
agaaccgctcgagcagacttgtctaaacgc

SEQ ID NO: 60 - DNA, 36 bp, Artificial Sequence
  sense primer
gtctgctcgagcggttctggaggaggagatatgagg

SEQ ID NO: 61 - DNA, 30 bp, Artificial Sequence
  antisense primer
acgtcctgcagacttggtgggtgctactcc